(19) United States
(12) Patent Application Publication
(10) Pub. No.: US 2026/0294467 A1
(43) Pub. Date: Oct. 1, 2026

(54) SELECTIVELY DILATABLE STENT HAVING ONE OR MORE CUTTING EDGES, AND RELATED SYSTEMS AND METHODS

(71) Applicant: Bard Peripheral Vascular, Inc., Franklin Lakes, NJ (US)

(72) Inventors: Zachary Kevin Willis, Mesa, AZ (US); Austin Taylor Jones, Mesa, AZ (US); Ryan Patrick Giudice, Gilbert, AZ (US); Greta Raeann Hull, Phoenix, AZ (US)

(21) Appl. No.: 19/089,287

(22) Filed: Mar. 25, 2025

(51) Int. Cl. A61B 17/3207; A61B 17/32

(52) U.S. Cl. CPC A61B 17/320725; A61B 2017/320052; A61B 2017/320716

(57) ABSTRACT

An example stent includes a stent body and one or more cutting edges. The stent body is expandable to a deployed configuration having an outer periphery. The one or more cutting edges extend outwardly relative to the outer periphery of the stent body when the stent body is in the deployed configuration.

### BACKGROUND

Post-thrombotic syndrome (PTS) is a serious condition that can develop in an individual after a deep vein thrombosis (DVT, e.g., a blood clot in a deep vein). The DVT in the vein may prevent blood from blowing back to the heart properly. Symptoms of PTS may include persistent leg pain, varicose veins, skin changes (e.g., discoloration, ulcers, and eczema), and/or leg fatigue and weakness. The clot causing the PTS also may harden over time, increasing the severity of the PTS and decreasing the likelihood of effective treatment of the clot/DVT with therapeutics.

A thrombectomy is a medical procedure to remove a blood clot from the blood vessel. Convention systems for removing clots, however, are limited in the strength required for a medical device to remove some clots, particularly those clots that have hardened over time.

### SUMMARY

Embodiments disclosed herein are related to selectively dilatable stents having one or more cutting edges, and related systems and methods. In an embodiment, a stent system includes a handpiece having one or more controls, a catheter assembly, and a stent. The catheter assembly includes an outer shaft secured to the handpiece and a delivery catheter positioned at least partially within the outer shaft and having a first lumen and a first distal end region distal to the handpiece. At least one of the delivery catheter or the outer shaft is selectively movable to move the first distal end region of the delivery catheter from a delivery position within the outer shaft to a deployment position distal to the outer shaft. The stent is secured to the delivery catheter. The stent is selectively movable with the delivery catheter between the delivery position with the stent disposed at least partially within the outer shaft and the deployment position with the stent disposed at least partially outside the outer shaft. The stent includes a stent body having an outer periphery and one or more cutting edges extending at least partially outwardly relative to the outer periphery of the stent body. The handpiece is configured to selectively rotate the stent body to rotate the one or more cutting edges within a vein.

In an embodiment, a method of treating a clot within a vein is disclosed. The method includes advancing a guidewire through the vein and the clot within the vein and inserting a catheter assembly of a stent system into the vein. The catheter assembly includes an outer shaft secured to the handpiece and a delivery catheter positioned at least partially within the outer shaft and having a first lumen and a first distal end region distal to the handpiece. The stent system includes a handpiece secured to the catheter assembly and a stent disposed within the outer shaft in a collapsed configuration, the guidewire extending through the catheter assembly. The method also includes advancing at least a portion of the outer shaft in the vein distally past the clot in the vein and at least partially retracting the outer shaft such that the stent is exposed within the vein in a deployment position. The method also includes expanding a stent body of the stent to a first deployed configuration such that one or more cutting edges extending outwardly relative to an outer periphery of the stent body in the first deployed configuration contact the clot. The method also includes rotating the stent body to cut or dislodge one or more portions of the clot in the vein.

In an embodiment, a stent includes a stent body that is expandable to a deployed configuration having an outer periphery and one or more cutting edges extending outwardly relative to the outer periphery of the stent body when the stent body is in the deployed configuration.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a perspective view of a stent system, according to an embodiment.

FIG. 2A is a partial cross-sectional view of a catheter and a stent of the stent system of FIG. 1, according to an embodiment.

FIG. 2B is a cross-sectional view of the stent of the stent system of FIG. 1, according to an embodiment.

FIGS. 3A-3F are partial side cross-sectional views of the catheter and stent of the stent system of FIG. 1 during deployment stages of the stent in a vein and removal stages of a lesion or clot in the vein with the stent, according to an embodiment.

FIG. 4 is a side view of a stent, according to an embodiment.

FIG. 5 is a flow diagram of a method of treating a clot within a vein, according to an embodiment.

### DETAILED DESCRIPTION

Embodiments disclosed herein are related to selectively dilatable stents having one or more cutting edges, and related systems and methods. In many embodiments, the stents disclosed herein may be used for treatment of acute and/or subacute lesions or clots within the venous vasculature. Conventional stents for removing clots typically lack the strength sufficient to remove some clots, such as older, more hardened or fibrous clots. In at least one, some, or all embodiments of the selectively dilatable stents disclosed herein, the stent is constructed in a cross-linked diamond or argyle-like structure with one or more struts or supports disposed longitudinally on an outside of the stent. Each of the one or more struts includes a cutting or slicing edge thereon or secured thereto according to at least one, some, or all embodiments. For example, the cutting edges may be mounted on the side of the stent as sharpened structures that cut and/or scoop the lesions or clots with an incorporated cutting edge. In certain embodiments, the cutting edge may have a depth (e.g., maximum distance between the cutting edge and the stent) of about 1 mm to about 3 mm.

In use of many embodiments of the selectively dilatable stents of this disclosure, a guidewire may be fed through a vessel past the lesion or clot. A proximal end of the stent may be attached to a delivery catheter and a distal end of the stent may be attached to a sliding catheter. In some embodiments, an embolic protection device (EPD) is attached to at least one of the catheter or the stent distal to the stent. Linkages on tendrils of the EPD may allow for deployment and retrieval of the EPD using a sheath. Upon delivery and alignment of the stent, an outer shafter may be pulled back proximally to deploy the EPD downstream from the lesion or clot. The cross-linked diamond or argyle-like structure of the stent allows the stent to be self-deployed from a catheter. In some embodiments, the stent may be self-deployed by removing a constraining device (e.g., outer catheter or sheath) or utilizing one or more shape memory materials (e.g., nitinol) to self deploy at a body temperature of the patient. The stent body may be selectively expanded until the stent body contacts at least a portion of the lesion or clot. Once a selected diameter of the stent body is achieved, the diameter of the stent body may be locked. A practitioner may then selectively rotate the stent body by rotating a handpiece, with the cutting edges separating the lesion or clot from the wall of the vein. In many embodiments, after an initial rotation and cutting of the lesion, the stent body may be further expanded to remove additional lesions or clot material from the stent wall. Throughout this process the structure of the stent including the cross-linked diamond structure of the stent body, the struts, and the cutting edges provides improved strength sufficient to remove more hardened or fibrous lesions or clots.

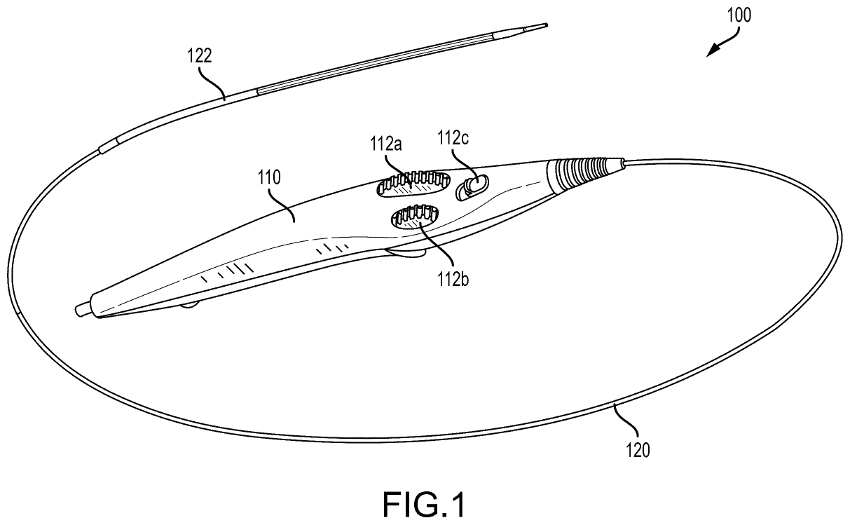

Turning to the drawings, FIG. 1 is a perspective view of a stent system 100, according to an embodiment. The stent system 100 may include a handpiece 110 having one or more controls 112a-c and a catheter assembly 120 including an outer shaft 122 secured directly or indirectly to the handpiece.

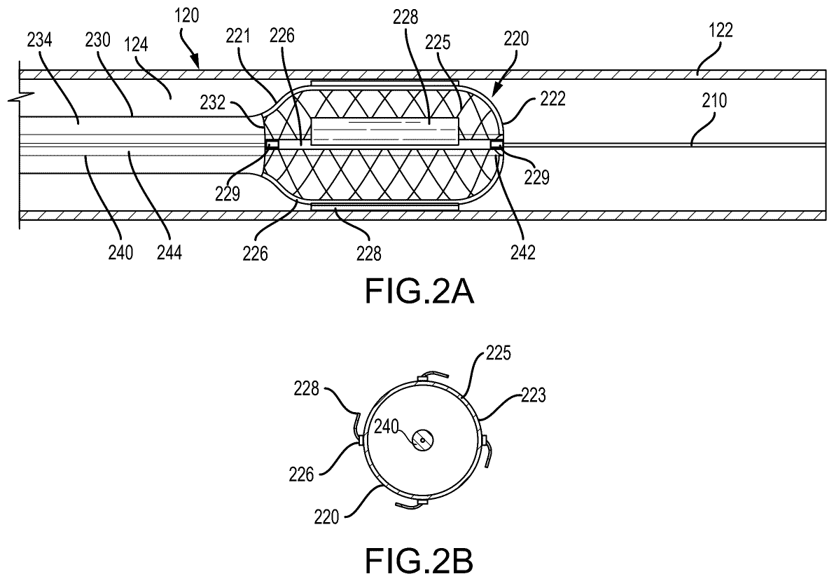

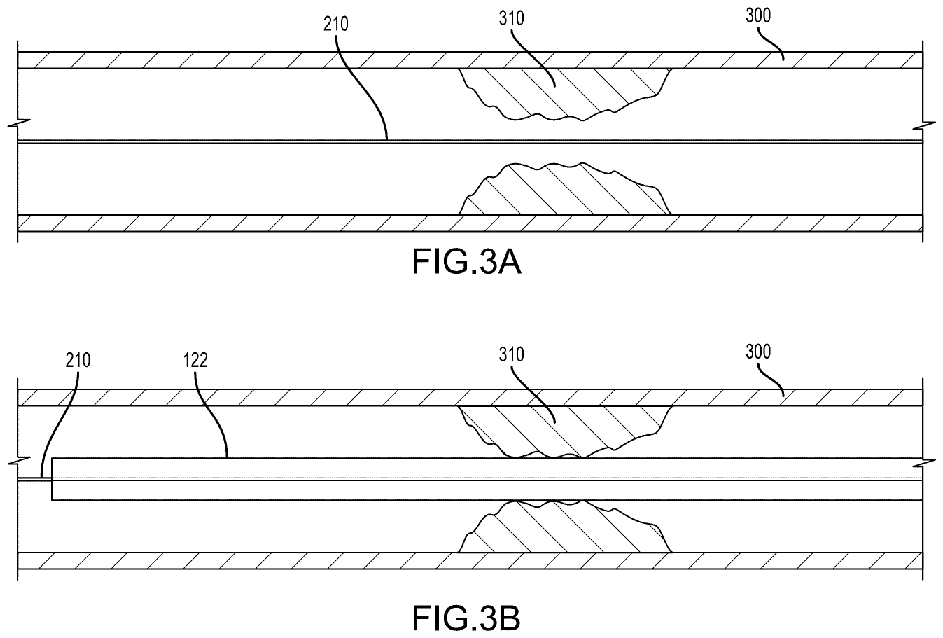

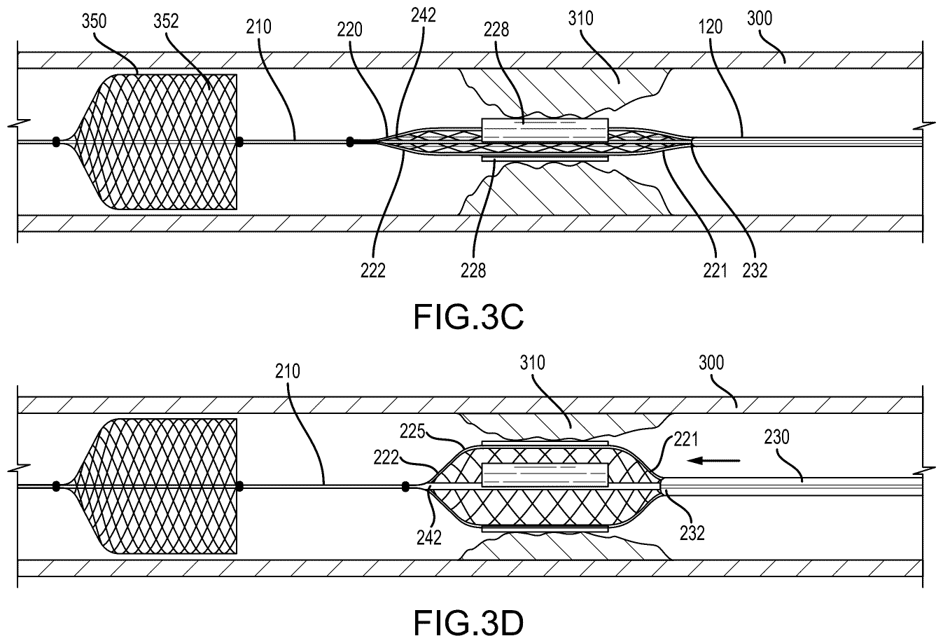

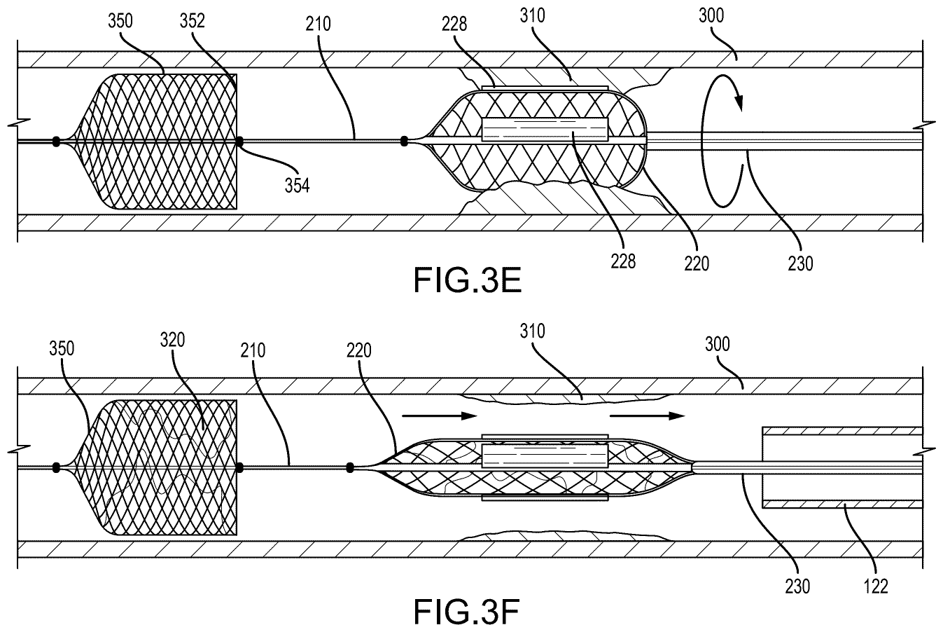

Turning ahead in the drawings, FIG. 2A is a partial cross-sectional view of the catheter assembly 120 and a stent 220 of the stent system 100. The catheter system may include the outer shaft 122 defining a shaft lumen 124, a delivery catheter 230, and an inner catheter 240. The stent system 100 also may include a guidewire 210 extend at least partially through the stent 220 and/or a lumen 244 of the inner catheter 240. The delivery catheter 230 is positioned at least partially within the shaft lumen 124 of the outer shaft 122 and includes a first lumen 234 and a distal end region 232 distal to the handpiece 110. At least one of the delivery catheter 230 or the outer shaft 122 is selectively movable to move the first distal end region 232 of the delivery catheter 230 and the stent 220 from a delivery position (shown in FIG. 2A) within the shaft lumen 124 of the outer shaft 122 to a deployment position (shown in FIGS. 3C-3D) distal to the outer shaft 122. For example, at least one of the one or more controls 112a-c may at least partially retract the outer shaft 122 and/or at least partially extend the delivery catheter 230 to move the distal end region 232 and the stent 220 from the delivery position to the deployment position. The inner catheter 240 is disposed at least partially within the first lumen 234 of the delivery catheter 230, according to an embodiment. The inner catheter 240 may include a second distal end region 242 distal to the handpiece 110 and the first distal end region 232 of the delivery catheter 230.

The stent system 100 also includes the stent 220 secured to the distal end region 232 of the delivery catheter 230, according to an embodiment. In some embodiments, the stent 220 may include anchor struts (not shown) secured to the delivery catheter 230 (e.g., the distal end region 232 of the delivery catheter 230). For example, the anchor struts may be bonded to the delivery catheter 230 by bonding the anchor struts between a sleeve of plastic welded or otherwise secured to the delivery catheter 230. The anchor struts may be welded to the stent 220 or may be continuations of members of the stent 220 itself. In some embodiments, the stent 220 may be secured to the delivery catheter with one or more of an adhesive or a press-fit collar. The stent 220 may be selectively movable with the delivery catheter 230 between the delivery position with the stent 230 disposed at least partially within the shaft lumen 124 of the outer shaft 122 and the deployment position with the stent 220 disposed at least partially (e.g., entirely) outside the shaft 124 of the outer shaft 122. In some embodiments, the stent 220 has radiopaque markers 229 for visualization on each of the proximal end region 221 and the distal end region 222.

FIG. 2B is a cross-sectional view of the stent 220 of the stent system of FIG. 1, according to an embodiment. The stent 220 includes a stent body 225 having an outer periphery 223. The stent body 225 may include a generally diamond or argyle-like structure. For example, the material or strands of the stent body may be cross-linked to form a generally diamond or argyle-like structure. However, other suitable strut geometries may be employed. In some embodiments, the stent body 225 may include a wall stent structure. In some embodiments, the stent body 225 may be formed from a nickel-titanium superelastic alloy (e.g., nitinol) or other superelastic material which allows the stent body 225 to self-expand and/or to deploy when an external compressive force is removed (e.g., the outer shaft 122 is removed from the stent body 225). In some embodiments, the stent body 225 may be formed of one or more of nylon, stainless steel, polyurethane, metallic alloys, polymers, or combinations thereof. In some embodiments, such as a stent 220 that is expandable responsive to a balloon deployment, the stent body 225 may include other material(s), such as stainless steel. Descriptions and aspects of stent bodies (e.g., tubular prosthesis) are disclosed in U.S. patent application Ser. No. 15/966,120, filed on Apr. 30, 2018, the disclosure of which is incorporated herein, in its entirety, by this reference.

The stent 220 includes one or more cutting edges 228 extending outwardly relative the outer periphery 223 of the stent body 225. In some embodiments, the stent 220 includes one or more struts 226 disposed on the outer periphery 223 of the stent body 225, with the one or more cutting edges 228 being secured to the one or more struts 226. For example, the one or more struts 226 may extend longitudinally along the outer periphery 223 of the stent body 225. The one or more cutting edges 228 extend longitudinally the entire length of the one or more struts 226 or may extend longitudinally only partially along the one or more struts 226. The one or more cutting edges 228 may extend outward relative to the outward periphery 223 such that the terminating edge of the one or more cutting edges 228 is spaced from the outer periphery 223 of the stent body 225.

The one or more cutting edges 228 may include a sharpened terminating edge configured to slice or cut the lesion or clot in the vein. The one or more cutting edges 228 include a sharpened edge and may comprise or consist essentially of one or more of a metal (e.g., stainless steel, titanium, a metal alloy or nickel and titanium (nitinol)) or rigid plastics (e.g., nylon or polyoxymethylene). In some embodiments, the one or more cutting edges 228 may be electropolished to form the sharpened cutting edge 228. In many embodiments, a depth (e.g., maximum distance between the cutting edge 228 and the stent 220) of the one or more cutting edges is about 1 mm to about 5 mm, about 1 mm to about 3 mm, about 2 mm to about 4 mm, about 3 mm to about 5 mm, about 1 mm, about 2 mm, about 3 mm, about 4 mm, or about 5 mm. In some embodiments, the cutting edge 228 may include a serrated edge having, for example, one or more or relatively small serrations, larger scalloped serrations, single point serrations, or cuticle trimmer-sized serrations.

In some embodiments, the stent 220 includes a plurality of struts 226 and a corresponding plurality of cutting edges 228. For example, the stent 220 includes four struts 226 and four cutting edges 228. Other embodiments may include two to five struts 226 and corresponding cutting edges 228. The plurality of struts 226 (and the plurality of cutting edges 228) may be disposed substantially equal distances from one another. For example, struts of the four struts 226 are disposed approximately 90 degrees from adjacent struts on the stent 220, and cutting edges of the four cutting edges 228 are disposed approximately 90 degrees from adjacent cutting edges on the stent 220. The handpiece 110 may be configured to selectively rotate the stent body 225 to rotate the one or more cutting edges 228 within a vein. For example, rotating the handpiece 110 about an axis of the handpiece 110 may rotate one or more of the inner catheter 240 or the delivery catheter 230 to rotate the stent body 225.

In some embodiments, the one or more struts 226 and the one or more cutting edges 228 secured to the one or more struts 226 may be absent and the stent body 225 may itself be electropolished (e.g., on the diagonal strands forming the diamond structure of the stent body 225) such that the stent body 225 itself includes cutting edges configured to cut clot in more than just a rotational manner. In some embodiments, in addition to electropolishing, cutting edges may be manually sharpened or honed to create an intrinsic cutting mechanism on the stent body 220.

In many embodiments, the stent body 225 is selectively expandable between a collapsed configuration and one or more expandable configurations. At least one of the one or more controls 112a-c on the handpiece 110 may be configured to selectively expand the stent body 225 to the deployed configuration and collapse the stent body 225 to the collapsed configuration. Returning to FIG. 2A, the stent body 225 may include a stent proximal end region 221 secured to the first distal end region 232 of the delivery catheter 230 and a stent distal end region 222 secured to the second distal end region 242 of the inner catheter 240. The inner catheter 240 may be selectively moveable within the first lumen 234 of the delivery catheter 230. In these and other embodiments, at least one of the one or more controls 112a-c is configured to increase and/or decrease a distance between the first distal end region 232 of the delivery catheter 230 and the second distal end region 242 of the inner catheter 240 to expand (e.g., increase) and/or collapse (e.g., decrease) a diameter of the stent body 225. As the distance between proximal end region 221 and the distal end region 222 of the stent body 225 decreases, the diameter of the stent body 225 increases. As the distance between the proximal end region 221 and the distal end region of the stent body 225 increase, the diameter of the stent body 225 decreases. In some embodiments, this increase in distance may be achieved through use of a worm drive gear attached to the thumbwheel 112a or 112b of the handpiece 110 and a screw threaded spindle mounted to the delivery catheter 230.

For example, at least one of the one or more controls 112a-c may be configured to increase the distance between the first distal end region 232 of the delivery catheter 230 and the second distal end region 242 of the inner catheter 240. This increasing of the distance between the first distal end region 232 of the delivery catheter 230 and the second distal end region 242 of the inner catheter 240 also increases the distance between the proximal end region 221 and the distal end region 222 of the stent body 225 effective to collapse the stent body 225 (e.g., decrease a diameter of the stent body 225) to the collapsed configuration. At least one of the one or more controls 112a-c may be configured to decrease a distance between the first distal end region 232 of the delivery catheter 230 and the second distal end region 242 of the inner catheter 240. This decreasing of the distance between the first distal end region 232 of the delivery catheter 230 and the second distal end region 242 of the inner catheter 240 also decreases the distance between the proximal end region 221 and the distal end region 222 of the stent body 225 effective to expand the stent body (e.g., increase a diameter of the stent body 225) to a first deployed configuration. In many embodiments, at least one of the one or more controls 112a-c is configured to further decrease the distance between the first distal end region 132 of the delivery catheter 130 and the second distal end region 142 of the inner catheter 140. This further decreasing of the distance between the first distal end region 232 of the delivery catheter 230 and the second distal end region 242 of the inner catheter 240 also decreases the distance between the proximal end region 221 and the distal end region 222 of the stent body 225 effective to expand the stent body 225 (e.g., increase a diameter of the stent body 225) to a second deployed configuration having a greater diameter than the first deployed configuration.

Turning ahead in the drawings, FIGS. 3A-3F are partial side cross-sectional views of the catheter assembly 120 and stent 220 of the stent system 100 of FIG. 1 during deployment stages of the stent 220 in a vein 300 and removal stages of a lesion or clot 310 in the vein 300 with the stent 220, according to an embodiment. As illustrated in FIG. 3A, the guidewire 210 of the stent system 100 may be advanced distally through a clot 310. The outer shaft 122 of the catheter assembly 120 may be inserted or advanced distally through the clot 310, as illustrated in FIG. 3B. When the outer shaft 122 is initially advanced distally through the clot 310, the stent 220 may be positioned within the shaft lumen 124 of the outer shaft 122.

Turning ahead to FIG. 3C, the outer shaft 122 may be retracted proximally relative to the stent 220, the delivery catheter 230, and/or the inner catheter 240 to expose the stent 220. When the outer shaft 122 is retracted, the stent 220 may already be at least partially positioned within the clot 310. In some embodiments, the stent system 100 further includes a collection bag 350 (e.g., an embolic protection device (EDP)) secured or securable to the stent body 225. The collection bag 350 may include an open end oriented towards the stent body 225 and configured to collect clot material dislodged by the one or more cutting edges 228. In other words, the open end of the collection bag 350 is downstream from the stent 220 and oriented towards the distal end region of the stent 220. In some embodiments, the collection bag 350 is self-expanding, and may include a bag body 352 having any of the materials or configurations of the stent body 225. In some embodiments, the collection bag 350 is housed or held within the outer shaft 122 until the outer shaft 122 is retracted proximally to deploy the collection bag 350 in the vein downstream from the clot 310 and the stent 220. For example, the collection bag 350 may include a mesh bag that is placed on the guidewire 210 and encapsulated inside outer shaft 122. After advancing through the clot 310, the outer shaft 122 may be pulled back to reveal the mesh bag of the collection bag 350 with a windsock-like effect, connecting from tangent point on the guidewire 210 to the tip of the end of the collection bag 350. This collection bag 350 on the guidewire 210 may then be used as the guidewire for the stent 220. In some embodiments, connecting linkages on each tendril of the collection bag 350 allow for deployment and retrieval of the collection bag 350 using the outer shaft 122 or sheath.

Turning ahead to FIG. 3D, the delivery catheter 230 may be advanced distally while the inner catheter 240 remains substantially in place to deploy the stent 220 to at least a first deployed configuration. Accordingly, in some embodiments, this distal advancement of the of the delivery catheter 230 shortens the distance between the distal end region 232 of the delivery catheter 230 secured to the proximal end region 221 of the stent 220 and the distal end region 242 of the inner catheter 240 secured to the distal end region 222 of the stent 220. As provided above, this action of the delivery catheter 230 relative to the inner catheter 240 may increase a diameter of the stent 220.

The stent 220 may deployed to expand the stent body 225 until at least one of the stent body 225 or the one or more cutting edges 228 makes contact with the lesion or the clots 310. When the desired diameter of the stent 220 is reached (e.g., at least one of the stent body 225 or the one or more cutting edges makes contact with the lesion or the clots 310), the practitioner may stop advancing the delivery catheter 230 distally. In some embodiments, at least one of the one or more controls 112a-c on the handpiece may be used to advance the delivery catheter 230 proximally while the inner catheter 240 remains substantially stationary. In some embodiment, the stent 220 may be deployed by a screw drive that expands the stent body 225 until contact is made with the lesion or clot 310. For example, the stent system 100 may include a screw drive configured to actuate similarly to a scissor jack, e.g., with two jacks being set perpendicular to one another to fully dilate the stent. For example, the catheter assembly 120 may include a central catheter having a threaded exterior and configured to be rotated at the handle to actuate a nut attached to the two jacks. As the nut is moved in one direction, the nut may move a slide on a rail. A spring jack attached to both sides of this slide may be compressed or extended when the nut moves. A distal end of the spring jack may be fixed in place to create tension when compressed. When compressed, the spring jack may expand (increase in outer diameter). When extended, the spring jack may lay flat and reduce the outer diameter. In some embodiments, the nut may be secured to a proximal end of the threaded central catheter and one or more of the two jacks, slide, and/or spring jack may be positioned within the handpiece 110. Once appropriate diameter of the stent 220 is achieved, the diameter of the stent 220 may be locked with one or more of the controls 112a-c (e.g., 112c). In some embodiments, expansion of the stent 220 may be controlled at the handpiece 110 where the practitioner may increase or decrease the diameter of the stent 220 using a dial (e.g., 112a or 112b) and a worm drive gear connecting to the screw drive.

Turning ahead in the drawings to FIG. 3E, once the stent 220 is at a desired diameter (e.g., in a first deployed or expanded configuration), the practitioner may rotate the stent 220 to rotate the one or more cutting edges 228 effective to separate at least some of the clot 310 from the remainder of the clot 310 and/or the wall of the vein 300. In some embodiments, for example, the practitioner may rotate the stent 220 approximately 90 degrees to cut or dislodge at least some of the clot 310 from the remainder of the clot 310 and/or the wall of the vein 300. The practitioner may rotate the stent 220 by rotating the handpiece 110. For example, the stent 220 and the catheter assembly 120 may be secured to the handpiece 110 such that rotating the handpiece 110 rotates the stent 120 a corresponding amount. In other words, a practitioner may rotate the handpiece 110 approximately 90 degrees in a direction, for example, and the stent 220 may rotate a corresponding approximately 90 degrees in the same direction. In many embodiments, the stent system 100 includes a bearing 354 that allows the stent to rotate independent of the distal collection bag 350. After an initial pass, the diameter of the stent 220 may be further increased (e.g., to a second deployed or expanded configuration) and the stent 220 may again be rotated to remove additional clot 310.

Turning ahead in the drawings to FIG. 3F, the collection bag 350 with the open end 352 downstream from the stent 220 and the clot 310 may catch material 320 of the clot 310 cut or dislodged by rotation of the one or more cutting edges 228 on the stent 220 against the clot 310. In some embodiments, the stent 220 also may collect material 320 of the clot 310 cut or dislodged by rotation of the one or more cutting edges 228 on the stent 220 against the clot 310. The stent 220 may be relaxed, for example, with or without the trapped cut material 320 of the clot 310 inside and then retracted into the outer shaft 122 of the catheter assembly 120. The collection bag 350 also may be retracted into the outer shaft 122 of the catheter assembly 120.

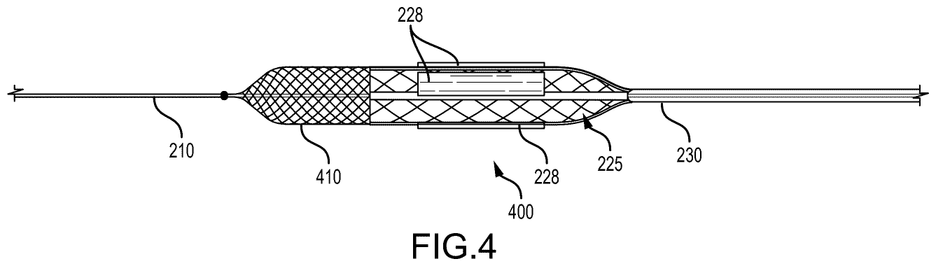

Turning ahead in the drawings, FIG. 4 is a side view of a stent 400, according to an embodiment. Unless otherwise notes, the stent 400 may include any aspect of the stent 220 and may be configured for use with the stent system 100 as described in relation to the stent 220. In some embodiments, the stent 400 may include an incorporated collection bag 410 (e.g., nitinol collection bag) rather than the tether collection bag 350. The incorporated collection bag 410 may be woven into the structure of the stent body 225 and may include an open end oriented towards the one or more cutting edges 228. In other words, the open of the of the bag 410 is oriented towards the distal end region of the stent 400. The incorporated collection bag 410 may include a mesh or tightly woven nitinol braided hemispherical cap on the stent body 225. In some embodiments, the incorporated collection bag 410 includes the same material as the stent body 225. In these and other embodiments, the cutting edge 228 may include an opening beneath the cutting edge 228 positioned and oriented to direct dislodged material within the stent 220 to be captured by the bag 410.

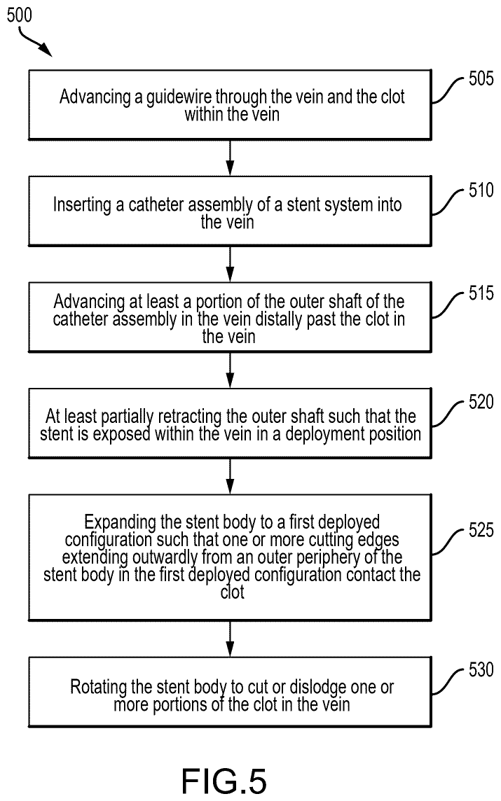

Turning ahead in the drawings, FIG. 5 is a flow diagram of a method 500 of treating a clot (e.g., lesion) within a vein, according to an embodiment. The method 500 may include use of any of the stent systems 100 or stents 220, 400 disclosed herein. In many embodiments, the method 500 encompasses one or more aspect of the deployment stages of the stent 200 in the vein 300 and removal stages of the clot 310 in the vein 300 with the stent 220 shown in FIGS. 3A-3F, and reference to these illustrations may be made in relation to the method 500. Acts of the method 500 are for illustrative purposes. For example, the acts of the method 500 may be performed in different orders, split into multiple acts, modified, supplemented, or combined.

In an embodiment, the method 500 includes advancing 505 a guidewire through the vein and the clot within the vein (e.g., as illustrated in FIG. 3A and accompanying description) and inserting 510 a catheter assembly of a stent system into the vein. The catheter assembly of the method 500 may include an outer shaft secured to the handpiece and a delivery catheter positioned at least partially within the outer shaft and having a first lumen and a first distal end region distal to the handpiece. The stent system of the method 500 may include a handpiece secured to the catheter assembly and a stent disposed within the outer shaft in a collapsed configuration, with the guidewire extending through the catheter assembly. The method 500 may include advancing 515 at least a portion of the outer shaft in the vein distally past the clot in the vein (e.g., as illustrated in FIG. 3B and accompanying description) and at least partially retracting 520 the outer shaft such that the stent is exposed within the vein in a deployment position (e.g., as illustrated in FIG. 3C and accompanying description). The method 500 also may include expanding 525 a stent body of the stent to a first deployed configuration such that one or more cutting edges extending outwardly relative to an outer periphery of the stent body in the first deployed configuration contact the clot (e.g., as illustrated in FIG. 3D and accompanying description). The method 500 also may include rotating 530 the stent body to cut or dislodge one or more portions of the clot in the vein (e.g., as illustrated in FIG. 3E and accompanying description).

In some embodiments, the method 500 also may include selectively collapsing the stent to the collapsed configuration and positioning the stent within the outer shaft (e.g., as illustrated in FIG. 3F and accompanying description).

In some embodiments of the method 500, the catheter assembly may include an inner catheter disposed at least partially within the first lumen of the delivery catheter and having a second distal end region distal to the handpiece the first distal end region of the delivery catheter. In these and other embodiments, the stent body may include a stent proximal end region secured to the first distal end region of the delivery catheter and a stent distal end region secured to the second distal end region of the inner catheter. Expanding 525 the stent body to a first deployed configuration such that one or more cutting edges extending outwardly relative to an outer periphery of the stent body in the first deployed configuration contact the clot may include, with the one or more controls, decreasing a distance between the first distal end region of the delivery catheter and the second distal end region of the inner catheter effective to expand the stent body to the first deployed configuration (e.g., as illustrated in FIG. 3D and accompanying description). The method 500 may further include expanding the stent body to a second deployed configuration having a greater diameter than the first deployed configuration by further decreasing the distance between the first distal end region of the delivery catheter and the second distal end region of the inner catheter.

In many embodiments of the method 500, the stent body includes a generally diamond structure with one or more struts disposed on the outer periphery of the stent body, the one or more cutting edges being secured to the one or more struts. In some embodiments, the stent body may include a woven or braided wire configuration. A depth of the one or more cutting edges is about 1 mm to about 3 mm in the method 500.

In many embodiments, the method 500 may further include collecting clot material dislodged or cut by the one or more cutting edges with at least one of the stent body or a collection bag secured or securable to the stent body. The collection bag may be secured directly to an end region of the stent body. The collection bag may be configured to not rotate while rotating the stent body to cut or dislodge one or more portions of the clot in the vein.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

What is claimed is:

1. A stent system, comprising:
a handpiece;
a catheter assembly including an outer shaft secured to the handpiece and a delivery catheter positioned at least partially within the outer shaft and having a first lumen and a first distal end region distal to the handpiece, at least one of the delivery catheter or the outer shaft selectively movable to move the first distal end region of the delivery catheter from a delivery position within the outer shaft to a deployment position distal to the outer shaft; and
a stent secured to the delivery catheter, the stent being selectively movable with the delivery catheter between the delivery position with the stent disposed at least partially within the outer shaft and the deployment position with the stent disposed at least partially outside the outer shaft, the stent including a stent body having an outer periphery and one or more cutting edges extending at least partially outwardly relative to the outer periphery of the stent body, wherein the handpiece is configured to selectively rotate the stent body to rotate the one or more cutting edges within a vein.

2. The stent system of claim 1, wherein:
the stent body is selectively expandable between a collapsed configuration and an expandable configuration; and
the handpiece includes one or more controls configured to selectively expand the stent body to the deployed configuration and collapse the stent body to the collapsed configuration.

3. The stent system of claim 2, further comprising:
an inner catheter disposed at least partially within the first lumen of the delivery catheter and having a second distal end region distal to the handpiece the first distal end region of the delivery catheter, wherein the inner catheter is selectively moveable within the first lumen of the delivery catheter;
wherein the stent body includes a stent proximal end region secured to the first distal end region of the delivery catheter and a stent distal end region secured to the second distal end region of the inner catheter;
wherein the one or more controls are configured to:
decrease a distance between the first distal end region of the delivery catheter and the second distal end region of the inner catheter effective to expand the stent body to a first deployed configuration; and
increase the distance between the first distal end region of the delivery catheter and the second distal end region of the inner catheter effective to collapse the stent body to the collapsed configuration.

4. The stent system of claim 3, wherein the one or more controls are configured to further decrease the distance between the first distal end region of the delivery catheter and the second distal end region of the inner catheter effective to expand the stent body to a second deployed configuration having a greater diameter than the first deployed configuration.

5. The stent system of claim 1, wherein the stent body has a generally diamond structure with one or more struts disposed on the outer periphery of the stent body, the one or more cutting edges being secured to the one or more struts.

6. The stent system of claim 1, wherein a depth of the one or more cutting edges is about 1 mm to about 3 mm.

7. The stent system of claim 1, further comprising a collection bag secured or securable to the stent body, the collection bag having an open end oriented towards the stent body and configured to collect clot material dislodged by the one or more cutting edges.

8. The stent system of claim 7, wherein the collection bag is secured directly to an end region of the stent body.

9. The stent system of claim 7, wherein the stent body is configured to rotate while the collection bag does not rotate.

10. The stent system of claim 1, wherein the one or more cutting edges comprises a plurality of cutting edges spaced at equal distances from one another on the outer periphery of the stent body.

11. A stent, comprising:
a stent body that is expandable to a deployed configuration having an outer periphery; and
one or more cutting edges extending outwardly relative to the outer periphery of the stent body when the stent body is in the deployed configuration.

12. The stent of claim 11, wherein the stent body is selectively expandable between a collapsed configuration and an expandable configuration.

13. The stent of claim 1, wherein the stent body has a generally diamond structure with one or more struts disposed on the outer periphery of the stent body, the one or more cutting edges being secured to the one or more struts.

14. The stent of claim 1, wherein a depth of the one or more cutting edges is about 1 mm to about 3 mm.

15. The stent of claim 1, further comprising a collection bag secured or securable to the stent body, the collection bag having an open end oriented towards the stent body and configured to collect clot material dislodged by the one or more cutting edges.

16. The stent of claim 15, wherein the collection bag is secured directly to an end region of the stent body.

17. The stent of claim 11, wherein the one or more cutting edges comprises a plurality of cutting edges spaced at equal distances from one another on the outer periphery of the stent body.

18. A method of treating a clot within a vein, the method comprising:
advancing a guidewire through the vein and the clot within the vein;
inserting a catheter assembly of a stent system into the vein, the catheter assembly including an outer shaft secured to the handpiece and a delivery catheter positioned at least partially within the outer shaft and having a first lumen and a first distal end region distal to the handpiece, the stent system including a handpiece secured to the catheter assembly and a stent disposed within the outer shaft in a collapsed configuration, the guidewire extending through the catheter assembly;
advancing at least a portion of the outer shaft in the vein distally past the clot in the vein;
at least partially retracting the outer shaft such that the stent is exposed within the vein in a deployment position;
expanding a stent body of the stent to a first deployed configuration such that one or more cutting edges extending outwardly relative to an outer periphery of the stent body in the first deployed configuration contact the clot; and
rotating the stent body to cut or dislodge one or more portions of the clot in the vein.

19. The method of claim 18, further comprising selectively collapsing the stent to the collapsed configuration and positioning the stent within the outer shaft.

20. The method of claim 18, wherein:
the catheter assembly includes an inner catheter disposed at least partially within the first lumen of the delivery catheter and having a second distal end region distal to the handpiece the first distal end region of the delivery catheter;
the stent body includes a stent proximal end region secured to the first distal end region of the delivery catheter and a stent distal end region secured to the second distal end region of the inner catheter; and
expanding the stent body to a first deployed configuration such that one or more cutting edges extending outwardly relative to an outer periphery of the stent body in the first deployed configuration contact the clot includes, with one or more controls on the handpiece, decreasing a distance between the first distal end region of the delivery catheter and the second distal end region of the inner catheter effective to expand the stent body to the first deployed configuration.

21. The method of claim 20, further comprising expanding the stent body to a second deployed configuration having a greater diameter than the first deployed configuration by further decreasing the distance between the first distal end region of the delivery catheter and the second distal end region of the inner catheter.

22. The method of claim 18, wherein the stent body has a generally diamond structure with one or more struts disposed on the outer periphery of the stent body, the one or more cutting edges being secured to the one or more struts.

23. The method of claim 18, wherein a depth of the one or more cutting edges is about 1 mm to about 3 mm.

24. The method of claim 18, further comprising collecting clot material dislodged or cut by the one or more cutting edges with at least one of the stent body or a collection bag secured or securable to the stent body.

25. The method of claim 24, wherein the collection bag is secured directly to an end region of the stent body.

26. The method of claim 24, wherein the collection bag does not rotate while rotating the stent body to cut or dislodge one or more portions of the clot in the vein.
